(19) United States
(12) Patent Application Publication
(10) Pub. No.: US 2016/0095565 A1
(43) Pub. Date: Apr. 7, 2016

(54) METHOD AND IMAGING SYSTEM FOR COMPENSATING FOR LOCATION ASSIGNMENT ERRORS IN PET DATA OCCURRING DUE TO A CYCLICAL MOTION OF A PATIENT

(71) Applicant: Siemens Aktiengesellschaft, Muenchen (DE)

(72) Inventor: Matthias Fenchel, Erlangen (DE)

(73) Assignee: Siemens Aktiengesellschaft, Muenchen (DE)

(21) Appl. No.: 14/872,339

(22) Filed: Oct. 1, 2015

(30) Foreign Application Priority Data
Oct. 1, 2014 (DE) 102014219915.8

(51) Int. Cl. A61B 6/00; A61B 6/03; H04N 5/76

(52) U.S. Cl. CPC A61B 6/527; H04N 5/76; A61B 6/037; A61B 6/4417

(57) ABSTRACT

In a method for compensating for location assignment errors in PET data that occur due to a cyclical motion of a patient, three-dimensional training data of the patient are acquired with an image recording facility using a different modality from PET in different motion states of the cyclical motion. Model parameters of a statistical model are determined describing the cyclical motion, from the deviations of the training data in different motion states from displacement data describing a reference motion-state. A rule is determined for assigning measurement values of at least one measuring signal that can be recorded during the PET measurement process, and that describe motion states of the cyclical motion, to input parameters describing an instance of the statistical model. Measurement values are assigned to the PET data recorded for the respective recording time points. Displacement data for the PET data are determined using the assignment rule and the PET data are spatially displaced based on the displacement data.

### BACKGROUND OF THE INVENTION

1. Field of the Invention

The invention concerns a method for compensating for location assignment errors in PET data that occur due to a cyclical motion of a patient, in particular respiration, during a PET measurement process using a PET facility, as well as an image recording system and a storage medium encoded with programming instructions for implementing such a method.

2. Description of the Prior Art

During positron emission tomography (PET) a PET tracer is administered to a patient, the PET tracer accumulating at certain points within the body of the patient, for example in a tumor. The decay process of the PET tracer causes a positron to form, the annihilation of which by an electron produces two photons that move in precisely opposite directions. Two photons detected simultaneously by a PET detector describe what is referred to as a Line of Response (LOR), on which the event must have taken place. When a number of such PET events are recorded as PET data (PET raw data), sinograms result, from which it is possible to determine an image data record describing the spatial distribution of the PET tracer by back projection. The detector arrangement used here is generally a gantry, on which photodetectors that cover the entire 360° around the patient as completely as possible are arranged within a z coverage region.

In order to allow improved location assignment for PET tracer accumulations in an anatomical context, image recording systems (combination image recording facilities) have been proposed, which also include an image recording facility that uses a different modality in addition to the PET facility. These two imaging facilities are integrated with one another in some instances, but in any case are in registration with one another, so that PET data can be assigned to the image data of the other image recording facility. The other image recording facilities are usually magnetic resonance facilities or computed tomography facilities, so that combined MR/PET or CT/PET image recording systems result.

PET data are generally recorded over a long recording period, for example in the range of 2 to 10 or even 45 minutes. This time is much longer than the typical duration of a respiratory cycle of the patient and it is not possible for the patient to hold his/her breath for a correspondingly long time. A PET measurement may therefore be impaired by the respiratory motion (or in some instances also other cyclical motion of the patient, in particular the heartbeat). This is of course also true of other system-related motion, which should be prevented where possible, which is not possible with cyclical patient motion.

The cyclical motion, in particular the respiratory motion, produces artifacts in the PET image data record to be reconstructed, manifested as blurring of anatomical structures, corresponding to the movement path of the anatomical structure due to the motion. This makes it difficult to interpret the PET image data records and they cannot be quantified accurately, because boundaries are less sharp and the peak intensities of tracer accumulations are reduced. In order to improve reliability, reproducibility and quantifiability in PET imaging and to increase PET sensitivity even for smaller lesions, it is expedient to correct motion in the PET data. There are several known approaches to this in the literature.

In the prior art the respiratory cycle is generally subdivided into different respiratory phases; in other words similar motion states during an entire respiratory cycle are combined into respiratory phases, known as gates. This is generally based on respiratory amplitude. Applications that utilize such gating to calculate PT image data records for just one defined respiratory phase are therefore generally referred to as gating applications. In such applications the motion is ultimately “frozen” to the characterizing motion state for the respiratory phase at the cost of the signal to noise ratio, as only the samplings of an individual respiratory phase are included in the PET image data record.

The expression “motion correction” in the context of gating is used whenever a correction function or correction motion field is deployed for each respiratory phase, in other words a means for correcting the location assigned to each individual PET event to a reference location in a defined reference respiratory phase. A dense displacement vector field, which is determined in the context of a registration of an image data record of a respiratory phase with an image data record of the reference data phase, can be used here. Variants of such gating motion correction differ either in the modality of the image data records used as input data for determining the correction functions/correction motion fields (PET-based, MR-based, etc.) and/or in the manner in which the correction functions/correction motion fields are determined (registration by means of diffeomorphic demons, mass-conserving optical flow and the like). For such prior art reference can be made, for example, to the following articles:
[1] M. Dawood et al., “A mass conservation-based optical flow method for cardiac motion correction in 3D-PET”, Med. Phys. 2013, 40:012505-1-012505-9;
[2] R. Grimm et al., “Self-Gated Radial MRI for Respiratory Motion Compensation on Hybrid PET/MR Systems”, LNCS: MICCAI 2013, 8151:17-24;
[3] C. Würslin et al., “Respiratory motion correction in oncologic PET using T1-weighted MR imaging on a simultaneous whole-body PET/MR system”, JNM 2013, 54:464-471;
[4] B. Guérin et al., “Nonrigid PET motion compensation in the lower abdomen using simultaneous tagged-MRI and PET imaging”, Med. Phys. 2011, 38:3025-3038; and
[5] Se Young Chun et al., “MRI-Based Nonrigid Motion Correction in Simultaneous PET/MRI”, JNM 2012, 53:1-8.

Such embodiments can of course also be applied to cardiac motion, which however can generally be considered to be predictable in respect of amplitude and sequence.

A basic disadvantage of such gating approaches, which ultimately strive to combine similar motion states into one phase, in particular with respect to the patient's respiration, is the inherent assumption that the motion in question follows a discrete pattern and also adheres to this pattern in the future, thus assuming that cyclical motion can be represented by a few respiratory phases (“thin representation”). However in reality such an assumption is often not correct. In practice patients often breathe deeply and heavily at the start of the examination, breathing in a flatter manner later as they relax. In other instances the patient may breathe in a flatter manner at first, taking deeper breaths later when they feel less comfortable.

A further problem with respect to motion models that aim to model motion by combining motion states into phases, is that the lower limit of accuracy of freezing motion or the upper limit of achievable image sharpness of each of the gating methods is primarily determined by the corresponding properties of each of the individual such phases, as the resulting PET image data record is determined as a motion-corrected average of the images of all the phases.

Because measurements for motion modeling are auxiliary measurements with no diagnostic relevance of their own, they should generally take little time and allow the broadest possible use, in particular with regard to changes in respiratory pattern and later time points.

### SUMMARY OF THE INVENTION

An object of the present invention is to provide motion correction for PET data that allows higher PET image data record quality to be achieved.

According to the invention, a method of the general type described above has the following steps.

Three-dimensional training data for the patient are recorded (acquired) with an image recording facility using a different modality from PET in different motion states of the cyclical motion of the patient.

Model parameters of a statistical model describing the cyclical motion are determined in a processor from the deviations of the training data in different motion states from displacement data describing a reference motion state.

A rule is determined in the processor for assigning measurement values of at least one measuring signal that can be recorded during the PET measurement process, and that describe motion states of the cyclical motion to input parameters describing an instance of the statistical model.

Measurement values assigned to the PET data recorded for the respective recording time points are obtained during the PET measurement process.

Displacement data for the PET data are obtained in the processor using the assignment rule for the assigned measurement value and the statistical model.

The PET data are displaced in the processor spatially based on the displacement data, thereby producing corrected PET data that are made available in electronic form, as a data file, from the processor.

The present invention therefore provides a way of avoiding the weaknesses of discrete motion representation, by a generalizable, continuous motion model being used as the statistical model, correlated with measuring signals that also describe the cyclical motion. Such measuring signals or more specifically their measurement values can be supplied by additionally provided measuring facilities, but can also be made available by the PET facility or the image recording facility itself. It has proven to be the case that the use of statistical models for cyclical motion brings with it not only a significant reduction of the data to be stored compared with the gating approaches, but also results in a reduction of the dimensionality of the motion description, thereby allowing a fast or even ultrafast PET reconstruction, or even real time LOR rebinning based on the displacement data.

The reduction of the data to be stored already represents a major advantage, because less storage space is required. With the aforementioned gating approaches, a greater precision would require more phases, for example more respiratory phases, with the result that the storage of data for each phase would not only increase the storage space required but would also increase the utilization of data transmission paths and calculation times. An increase in the number of gates is also associated with a loss of quality due to a reduced signal to noise ratio or an increased susceptibility to artifacts, so that in practice high gate numbers do not mean an increase in accuracy.

Advantages also result from the fact that only very few input parameters are required for the statistical model, in order to be able to describe a defined motion state, because the cited at least one measuring signal for tracking the cyclical motion is generally also of small dimension. This in turn means that when determining the assignment rule, it is necessary to correlate one parameter set of small dimension with another parameter set of small dimension, thereby resulting in a better and more practical way of evaluating the similarity and equivalence of the input parameters and measuring signals. This is particularly the case when an optimum measuring signal is to be selected from a number of measuring signals.

By far the simplest way of determining the assignment rule here is to record the at least one measuring signal with the training data, with the assignment rule being determined as a function of the measurement values assigned to the respective motion states of the training data. Other approaches are in principle also conceivable but these require a further series of training measurements to determine the assignment rule and are therefore less preferable.

In a preferred embodiment, an image recording system is used with the PET facility and the image recording facility, with the coordinate systems of the image recording facility and the PET facility being in registration in the image recording system. This also has the advantage that the patient generally does not have to move between the recording of the training data and the PET measurement process. Using an image recording system (often also referred to as a combination image recording facility) has the major advantage that the displacement data derived from the training data can also easily be used in the coordinate system of the PET data, so that it is clear to which LOR the PET data should actually be assigned based on the displacement data. Exemplary embodiments are also possible, in which registration is not already present between the image recording facility and the PET facility; a further registration process is then required to apply the statistical model (which can also be referred to as the motion model) to the PET data. In this context however the motion state must be identifiable in the data records used for registration, which can be made possible by way of measurement values of the at least one measuring signal.

A magnetic resonance facility or a computed tomography facility can be used as the image recording facility, since image recording systems, which combine magnetic resonance and PET, or computed tomography and PET, are widely known. Of course combination with other image recording facilities, for example ultrasound facilities and the like, is also conceivable. It should be noted that it is in principle also conceivable to use the PET facility itself as the image recording facility, but this is less preferable due to the lesser data diversity (only the concentration of PET tracer is mapped), the poor temporal resolution, and the additional administration of PET tracer that is required in some instances.

The present method therefore starts with the recording of training data. The patient is scanned for motion modeling. Provision can be made for training data to be recorded for at least five different motion states by gating, but it is also conceivable and preferable for training data to be recorded in a four-dimensional manner. The result of each of these recordings is a set of three-dimensional training data records respectively for different motion states. During a gated recording gating in the manner of triggering based on the measuring signal, is conceivable but retrospective consideration is also possible, with the measuring signal again being used as the basis. It is preferable for temporally continuous, dynamic imaging to take place in order to record the training data; in other words three-dimensional data records are generated in fast succession and each of the data records is considered to map one motion state. For each of said training data records the measurement value is also expediently recorded for each of the at least one measuring signals.

It should also be noted that the reference motion state can ultimately be selected arbitrarily from the training data. For example the reference state can be a defined respiratory phase selected as the reference or a different randomly selected reference motion state, for example from a different recording, for example a breath holding recording.

To record the training data in the example of magnetic resonance or to some extent also with other modalities it is possible to use a very wide range of options, for example repeated breath holding in different motion states, repeated gated recordings with different amplitudes, dynamic approaches, in particular operating with subsampling in the k-space, for example compressed sensing and the like.

In another embodiment of the invention, the displacement data are determined as dense displacement vector fields for the voxels of the training data assigned to the motion states. Displacement vectors for all the voxels of the training data records contained in the training data in particular are present in the displacement vector fields. Corresponding options for determining such displacement vector fields are known for example from optical flow calculations; in a particularly advantageous embodiment of the present invention provision can specifically be made for the displacement vector fields to be determined as part of an elastic registration process between the training data of a motion state and the training data of the reference motion state, as it is known in the context of a registration where the image information has been displaced to. If a training data record is present for the reference motion state, the training data records for the other motion states contained in the training data should ultimately be registered with this reference training data record.

The displacement data, in particular the displacement vector fields, then serve as input data for determining a statistical motion model. Statistical models generally speaking represent ways of encoding large-dimension data of linear or non-linear processes efficiently and reducing their dimensionality (compressing). It should be noted that it can be assumed that linear modeling is possible, particularly in the case of respiratory motion. It is of course also conceivable in principle to consider non-linear modeling approaches, if the nature of the cyclical motion requires this.

In another embodiment of the present invention, in this context a principal component analysis is performed to determine the linear statistical model or a kernel principal component analysis for the deviation data is performed to determine the non-linear statistical model, with at least some of the principal components being used as model parameters and their weights as input parameters. The principal component analysis (PCA) is an algorithm for performing statistical modeling that has been known for some time. Principal components (primary components) with assigned intrinsic values are determined, the total of the intrinsic values indicating the relevance of the corresponding principal components, and it is frequently sufficient only to consider very few principal components with the greatest intrinsic values. If a linear combination of the principal components of the variation is used together with the average motion, all the interpolated and extrapolated motion states for a defined patient can be determined according to the statistical model. It should be noted that linear statistical models are generally based on input data Gaussian-distributed in multiple dimensions. If the input data, here the displacement data, deviates too much from this prerequisite, it may prove expedient to use a non-linear statistical model, with the principal component analysis using a kernel (kernel PCA) being recommended. In this embodiment a kernel is used initially to transform the input data in such a manner that it satisfies the cited condition for Gaussian distribution, with the standard PCA then being applied to the transformed input data. The transformation can take place implicitly.

It should also be noted that Gaussian distributions in multiple dimensions can also be used to calculate the probability that defined displacement data is part of the statistical model. This allows incorrect measurements of the training data to be revealed as outliers and control measurements for example can be performed to test the suitability of the statistical model. A threshold value can be determined for this probability, it being possible to reject defined motion states that are not part of the statistical model.

It should also be noted that when a gating approach is used to determine training data records of different motion states, which then correspond to respiratory phases, the contributions of each respiratory phase weighted by the number of measurements can also be considered.

As mentioned above, it is expedient if only a few principal components with the highest intrinsic values are used as part of the statistical model. Provision can be made in particular for only fewer than five, in particular fewer than three, principal components with the highest intrinsic values to be used as part of the statistical model. This significantly reduces the number of input parameters for generating displacement data as instances of the model, thereby allowing a small-dimension representation of the motion states, which has the advantages discussed above in respect of the similarly small-dimension measuring signals.

It is also conceivable for the number of principal components with the highest intrinsic values to be used in the statistical model to be selected as a function of the ratios of their intrinsic values to the sum of all the intrinsic values.

This can be explained in more detail using a specific example. The vector components of the dense displacement vector fields, which can be used for example as displacement data and therefore input data, can be seen as multi-dimensional vectors. A singular value decomposition of the covariance matrix of all these multi-dimensional vectors supplies the principal components; in other words the characteristic vectors of the covariance matrix with their corresponding intrinsic values, which are presorted in descending size order. The amount of variance encoded by each principal component can be determined as the ratio of its intrinsic value to the sum of all the intrinsic values. The reduction of dimensionality is achieved, as described above, by omitting principal components of the lowest priority, for example such that a defined fixed component of the variance of the input displacement data can be described by the statistical model. Instances in which one or two principal components are already sufficient to map the majority of the respiratory motion of a given patient are possible. Each interpolated motion state in the statistical model can be described by

m=mₘₑₐₙ+Φ*b,

where mₘₑₐₙ is the average motion state, Φ is the matrix of the principal components in columns, and b is the linear weight of the principal components. The variation of b supplies interpolated or extrapolated instances of motion states m, which are associated with the linear statistical model. Changes in the respiratory amplitude can therefore be dealt with easily, as the statistical model is able to extrapolate data.

A next basic step of the inventive method is now to correlate the principal components, more specifically their weights, with potential sources for motion tracking, using temporal correlation with the measuring signals. Provision can be made specifically and generally here for the assignment rule to be determined using a regression algorithm and/or a prediction model and/or a machine-learning algorithm. To perform the correlation therefore the input data present is preferably measurement values of the at least one measuring signal, which are assigned to the training data records, with the weights or weight factors of the principal components (or generally input parameters of the model) assigned to the training data records also being used. Since therefore measurement values and assigned input parameters of the statistical model are present for different time points, a functional relationship, the assignment rule, can be derived by regression. A number of methods known in principle in the prior art can be used, for example linear or non-linear prediction models, machine-learning algorithms or regression models. If for example R designates a regression model, R can be trained so that the regression error is minimized.

It is conceivable for a number of measuring signals, which supply measurement values describing the motion state, to be present. In an advantageous embodiment of the invention then, when there are measurement values present for different measuring signals, a correlation value describing the quality of the correlation of the measurement values of the measuring signals with the input parameters for the training data at least is determined, with the measuring signal with the correlation value showing the best correlation being used as the measuring signal to be evaluated for the PET measurement process. It is therefore possible then to use the measuring signal with the highest correlation to determine displacement data describing motion states for other measurement values. In another embodiment, when measuring signals are recorded continuously during the recording of the training data for motion states that are not detected or that are combined into one phase, missing input parameters are interpolated for comparison with the measurement values and taken into account when determining the correlation value. It is therefore possible to interpolate the input parameters, for example the weights of the principal components, over time, in order to improve the basis for comparison. Generally the input parameters for defined motion states, therefore defined simultaneously recorded measurement values, in the example of PCA, can be obtained in such a manner that the equation cited above can be resolved for the vector b such that the displacement data for the time of the corresponding training data record results. If measurement values of the measuring signal are also present between the motion states, for which training data records are present, an interpolation can take place between said motion states, for example in a linear manner over time. The continuously present measuring signal can thus be correlated with the continuously present weight of the principal components.

When performing a regression to determine the assignment rule a regression class can expediently be used as the correlation value. Such regression classes are frequently supplied anyway in the assigned algorithms at the same time as the assignment parameters parameterizing the assignment rule.

In another embodiment of the present invention, the correcting displacement of the PET data takes place in real time immediately after it has been measured. The reduced quantity of data and the small dimensionality mean that the statistical model can be calculated quickly so a real time correction of PET data can take place when a PET event is measured and the corresponding measurement value of the measuring signal is present. This means that correlated PET raw data relating to the cyclical motion, in particular to the respiratory motion, is already present at the end of the PET measurement process and can be used directly to reconstruct a PET image data record.

The use of different measuring signals is conceivable in the context of the present invention, with a preferred embodiment providing for a function of the measured data events in a predetermined data space, in particular in the sinogram space, to be determined as the measuring signal. In this embodiment the PET events therefore form a basis for determining a measuring signal, without further measuring facilities necessarily being required, in that their spatial/temporal distribution is evaluated. More specifically provision can be made for a number of measured PET events in a locationally fixed volume of the patient in one time interval to be used as the measuring signal. The volume here is expediently selected in particular as a thin slice and/or as a PET image element such that an accumulation of PET tracer within the patient is moved into and out of the volume by the cyclical motion. Ultimately it is therefore determined that temporally more or temporally fewer PET events are determined along a defined LOR, due to the motion of a tracer accumulation out of and into said LOR. However this gives a measuring signal which, when the input parameters are suitably selected, allows motion states of the cyclical motion, in particular of the respiratory motion, to be concluded. For an exemplary embodiment of this, see the article by F. Büther et al., “List Mode-Driven Cardiac and Respiratory Gating in PET”, JNM 2009, 50: 674 to 681. If sufficient correlation can be determined in the context of the determination of the assignment rule, no further measuring signals from additional facilities are required.

It is also conceivable to use other measuring signals, for example the measuring signal from a respiratory belt and/or a respiratory cushion/or a three-dimensional camera and/or a navigator scan recorded using a magnetic resonance facility as the image recording facility.

All these measuring signals share the fact that they are of small dimension, which is also true of the input parameters of the statistical model.

Generally the inventive method therefore presents non-discrete, continuous motion modeling, in particular in respect of respiratory motion, which can be described with less data and smaller dimensionality by means of a statistical model, in particular determined by PCA. This can result in greater accuracy of motion correction and the efficient encoding predicted by storage space requirement and dimensionality can also be used to perform analyses for the optimum correlation and regression training of independent measuring signals for tracking motion. A further advantageous area of deployment is the real time correction of PET events along motion-corrected LORs over time. In particular it is possible to perform the corresponding calculations by means of an integrated circuit (IC), which allows extremely fast calculations on uncomplicated hardware. It is therefore conceivable in particular to have motion-corrected PET sinograms already available to conclude the PET measurement process, it being possible to use these directly to reconstruct the PET image data record, avoiding a motion-corrected reconstruction. Of course it is in principle also conceivable to apply motion correction to the PET data during the reconstruction and so on.

In addition to the method, the present invention encompasses an image recording system, having an image recording facility and a PET facility in registration with the image recording facility, as well as a control computer configured to perform the inventive method. All the embodiments relating to the inventive method apply to the inventive image recording system, with which it is therefore possible to achieve the abovementioned advantages. In particular, the control computer is configured to activate the components of the image recording facility in an appropriate manner to record three-dimensional training data. The control computer is also configured to determine model parameters of the statistical model describing the cyclical motion, and to determine the assignment rule, and receives measurement values of the measuring signal during the PET measurement process. The control computer is also configured to determine displacement data for the PET data and to correct the PET data, in particular by spatial displacement according to the displacement data, and is configured to make the corrected PET data available as a data file.

The invention also encompasses a non-transitory, computer-readable data storage medium encoded with programming instructions that cause the inventive method to be performed when executed in a control computer of an imaging system, in which the storage medium is loaded. All of the above embodiments also apply to this aspect of the invention. The storage medium is a non-volatile data medium, for example a CD-ROM.

### BRIEF DESCRIPTION OF THE DRAWINGS

FIG. 1 is a flowchart of an exemplary embodiment of the inventive method.

FIG. 2 outlines the correction of PET data in accordance with the invention.

FIG. 3 shows an inventive image recording system.

### DESCRIPTION OF THE PREFERRED EMBODIMENTS

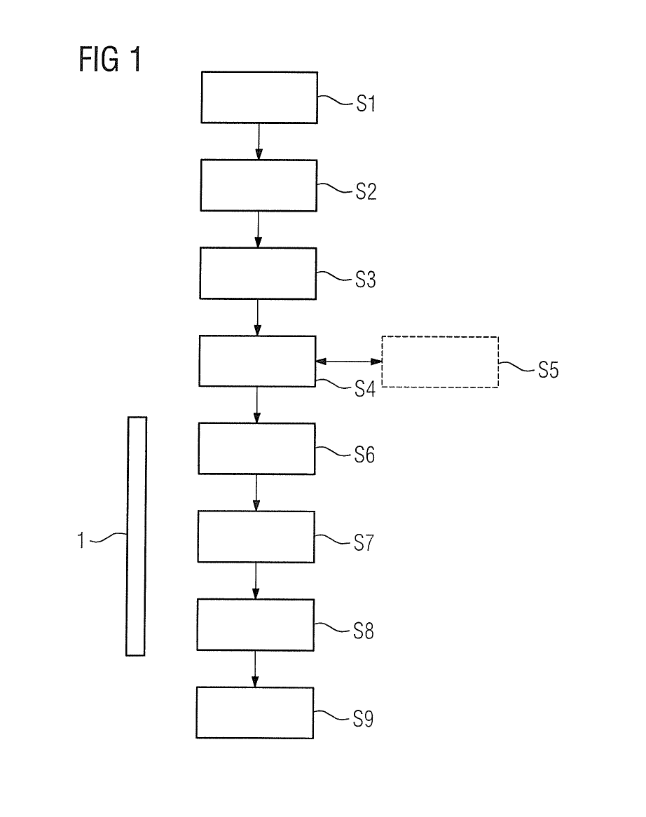

FIG. 1 is a flowchart of an exemplary embodiment of the inventive method. In the present instance it is performed on an image recording system, in which a magnetic resonance image recording facility and a PET facility are provided, being permanently in registration with one another. Different signals that describe a respiratory state of a patient, for whom a PET image data record is to be recorded, can be recorded, in particular a measuring signal from the PET facility itself, relating to the number of PET events in one time interval in a defined volume, the measuring signal from a respiratory belt and measuring signals from a navigator scan of the magnetic resonance facility.

In step S1, training data of the patient is first recorded, which shows the region of the patient affected by respiration in the form of three-dimensional training data records, each assigned to a defined motion state. The magnetic resonance facility is used to record the training data, with dynamic recording taking place over time in order to be able to pick up as many motion states as possible with one training data record. In order to increase temporal resolution a “compressed sensing” technique can be used to generate the then four-dimensional training data. It should be noted that it is also possible in principle to record using gating techniques but of course different motion states of the respiratory motion of the patient should be acquired.

Parallel to the training data in step S1 at least one measuring signal of the measuring signals referred to above is also recorded; it may be expedient to record a number of measuring signals, if a best correlating measuring signal is to be found later. In any case a measurement value of at least one measuring signal is or can be assigned to each motion state, in other words each training data record, after step S1.

In step S2, a reference motion state and therefore also a reference training data record is first selected from the training data. The aim here is to determine dense displacement vector fields as displacement data in respect of the reference motion state; in other words for each voxel of the training data records it is determined how said voxel has been displaced relative to the same feature shown in the reference training data record. This is done as part of the registration of the training data records of other motion states with the training data record of the reference motion state, as known in principle. The result is therefore a plurality of dense displacement vector fields, which indicate the extent to which voxels in other motion states have been displaced relative to a reference motion state.

In step S3, the training data should be compressed, in that a statistical model is determined therefrom, specifically model parameters of said statistical model. To this end the individual vectors of the dense displacement vector fields (three components per voxel) are interpreted as multi-dimensional vectors. In order to be able to perform a principal component analysis (PCA), a singular value decomposition of the covariance matrix of all said multi-dimensional vectors now takes place, giving as a result the principal components, in other words the characteristic vectors of the covariance matrix, and the associated intrinsic values, in descending size order. Linear combinations of the principal components (which form model parameters) with corresponding weighting factors and addition to the mean displacement vector field allow all the motion states of the training data to be mapped, as well as allowing intermediate motion states to be interpolated and motion states to be extrapolated outside the hitherto sampled region of the respiratory amplitude. It is frequently sufficient only to use a few principal components as part of the model, in particular fewer than three, for example one or two, principal components. In other words displacement vector fields, which are mapped by the linear statistical model, can generally be described by the average displacement vector field (model parameters) plus the sum of the principal components (model parameters) still being considered, multiplied by weighting factors (input parameters of the model).

It should also be noted that even though a linear statistical model has been presented here, it is in principle also conceivable to use non-linear statistical models, if the training data or displacement data requires this. For example a kernel PCA can be used. It should also be noted that the number of principal components retained can also be selected dynamically, for example by assessing their relevance using the ratio of the intrinsic value to the sum of all intrinsic values.

In step S4, in some instances together with an optional step S5, an assignment rule is determined, which can be used to determine weighting factors for defined measurement values of the at least one measuring signal, in other words input parameters of the statistical model, the assigned model instance therefore resulting as displacement data. As the weighting factors, in other words the input parameters for the displacement vector fields assigned to the training data records, can be determined easily, because they are known, pairs of measurement values and input parameters of the statistical model are therefore present for all the motion states of the training data being considered. This however allows the assignment rule to be determined by means of a regression algorithm, it being possible also, in some instances additionally, to deploy prediction models and/or machine-learning algorithms. It is also conceivable to interpolate interim values for the input parameters, in particular for discrete, separate motion states, for example using linear interpolation, in order thus to be able to increase the regression database, if measurement values of the at least one measuring signal were also recorded outside the motion states.

The optional step S5 relates to the situation where a number of measuring signals are considered. The regression in step S4 also supplies a regression class, which therefore describes how well the measurement values of the measuring signal and the input parameters of the statistical model correlate. In the following the measuring signal, for which the best correlation, therefore the highest correlation class, results, can be determined as the measuring signal. If only one measuring signal is considered, step S5 does not have to be performed of course.

The following steps S6-S8 take place during the PET measurement process indicated by the box 1. As part of step S6 the measuring signal is first recorded continuously during the PET measurement process. There are therefore always measurement values of the (in some instances best correlated) measuring signal present when a PET event occurs.

If this is the case, in step S7 the measurement value of the measuring signal recorded at the time point of the PET event is used to determine the assigned input parameters for the statistical model by means of the assignment rule. It is then possible to use the input parameters and the statistical model to determine displacement data, for example a displacement vector field again first, corresponding to the motion state determined by the measurement value.

Said displacement data determined in step S7 is used in step S8 in the present exemplary embodiment to perform a real time correction of the PET data of the PET event, as the displacement data shows displacement compared with the reference motion state along the corresponding LORs, as measured, so that it is possible to distribute the PET data of the PET event correspondingly to the adjacent, displaced LORs which are parallel, such that the probability of the PET event taking place in the displaced LORs, when it took place for the reference motion state, is satisfied.

As the statistical model includes an easily understandable number of model parameters and only a few or just one input parameter, in the present instance real time correction can be achieved by means of an integrated circuit, in other words a hardware module.

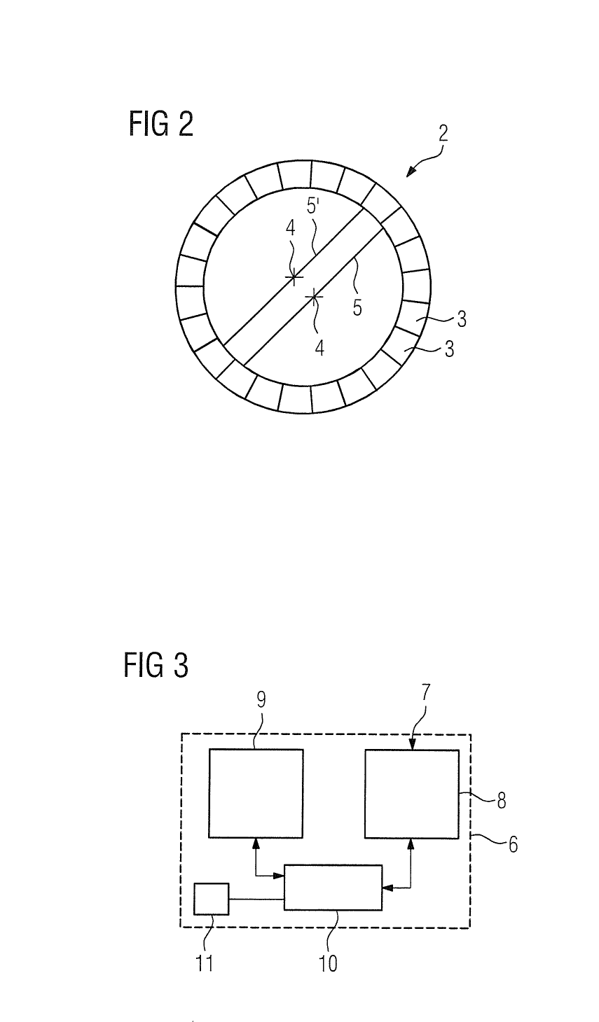

To explain said correction, reference is also made to the basic outline in FIG. 2, which shows a highly simplified representation of the PET gantry 2 with the photodetectors 3. When a PET event takes place at position 4, an LOR 5 results from the measurements of the photodetectors 3. Due to respiration the position 4 could however be displaced compared with the reference motion state, with the position 4 lying at position 4′. This results in a corresponding displaced LOR 5′.

As it is not known for a single PET event which point on the LOR 5 is the starting point of the PET event, a distribution is made to the parallel LORs 5′ as a function of how many positions 4 would be displaced to positions 4′ on the parallel LORs 5′.

It should also be noted that the inventive motion correction does not necessarily have to be performed as a real time correction. It is advantageous if the correction is performed in step S8 of FIG. 1, as there are then motion-corrected sinograms already present for the subsequent reconstruction of the 3D PET image data record. It is however also conceivable, in an alternative exemplary embodiment, to perform the motion correction during the reconstruction of the PET image data record in step S9.

FIG. 3 shows a basic outline of an inventive image recording system 6, which can be a combined MR/PET system. The image recording system 6 therefore has a recording facility 7 in the form of a magnetic resonance facility 8 and a PET facility 9. The PET facility 9 can be integrated at least partially into the magnetic resonance facility 8, for example in that the PET Gantry 2 is arranged in a patient accommodation region of the magnetic resonance facility 8. The PET facility 9 and the magnetic resonance facility 8 are registered with one another.

The operation of the PET facility 9 and the magnetic resonance facility 8 is controlled in the present instance by a control facility 10, which can also be divided up into control units for the individual image recording facilities 7, 9. The control facility 10 is configured by performing the inventive method.

The image recording system 6 can also comprise a measuring facility 11 for a measuring signal describing the respiratory motion of a patient, for example a respiratory belt, a respiratory cushion or the like.

Although modifications and changes may be suggested by those skilled in the art, it is the intention of the inventor to embody within the patent warranted hereon all changes and modifications as reasonably and properly come within the scope of his contribution to the art.

I claim as my invention:

1. A method for compensating for location assignment errors in the positron emission tomography (PET) data, comprising:
operating an image recording facility with a modality other than PET to acquire three-dimensional training data from a patient, exhibiting a cyclical motion, situated in the imaging recording facility, in a plurality of different motion states of the cyclical motion;
providing a processor with displacement data that describe a reference motion state and providing said processor with said training data and, in said processor determining model parameters of a statistical model that describes said cyclical motion, from deviations of said training data in said different motion states from said displacement data;
in said processor, determining an assignment rule for assigning measurement values of at least one measurement signal, which can be recorded during a PET measurement procedure and which exhibit said motion states of said cyclical motion, to input parameters that describe an instance of said statistical model;
operating a PET image recording facility to acquire PET data and to assign measurement values to the PET data acquired at respective recording points in time during a PET measurement procedure corresponding to the PET measurement procedure used to determine said rule;
in said processor, determining displacement data for said PET data by applying said assignment rule to said measurement values assigned to the PET data for said points in time; and
in said processor, spatially displacing said PET data according to said displacement data to obtain corrected PET data in which location assignment errors due to said cyclical motion are compensated, and making the corrected PET data available in electronic form, as a data file, from said processor.

2. A method as claimed in claim 1 comprising also recording at least one measuring signal with said training data and determining said assignment rule dependent on measurement values of said at least one measuring signal assigned to the respective motion states of the training data.

3. A method as claimed in claim 1 wherein said image recording system has a first spatial coordinate system associated therewith and wherein said PET imaging facility has a second spatial coordinate system associated therewith, and comprising, in said processor, electronically bringing said first and second coordinate systems into registration with each other.

4. A method as claimed in claim 1 wherein said image recording system has a first coordinate system associated therewith and wherein said PET image recording system has a second coordinate system associated therewith, and comprising integrating said image recording facility and said PET facility mechanically together with said first and second coordinate systems mechanically in registration with each other.

5. A method as claimed in claim 1 comprising employing, as said image recording facility, a facility selected from the group consisting of a magnetic resonance facility and a computed tomography facility.

6. A method as claimed in claim 1 comprising operating said recording facility to acquire said training data for at least five different motion states, by gating acquisition of said training data in the respective motion states.

7. A method as claimed in claim 1 comprising operating said image recording facility to acquire said training data as four-dimensional data.

8. A method as claimed in claim 1 comprising determining said displacement data as dense displacement vector fields for voxels of said training data assigned to said motion states.

9. A method as claimed in claim 8 comprising, determining said dense displacement vector fields by executing an elastic registration algorithm in said processor between said training data for a motion state and training data for the reference motion state.

10. A method as claimed in claim 1 comprising, in said processor, determining said statistical model by executing an algorithm selected from the group consisting of a primary component analysis algorithm to determine a linear statistical model as said statistical model, and a kernel primary component analysis to determine a non-linear statistical model as said statistical model, with at least some primary components being used as model parameters and weightings for said at least some of said principal components being used as input parameters.

11. A method as claimed in claim 10 comprising using at most five principal components as said model parameters that respectively have highest intrinsic values among all of the principal components.

12. A method as claimed in claim 10 comprising selecting a number of principal components used as said model parameters as being principal components, among all of the principal components, having highest intrinsic values dependent on respective ratios of the expected intrinsic values of the individual principal components to a sum of all intrinsic values of all of the principle components.

13. A method as claimed in claim 1 comprising determining said assignment rule by a procedure executed in said processor selected from the group consisting of execution of a regression algorithm, use of a prediction model, and execution of a machine-learning algorithm.

14. A method as claimed in claim 1 comprising, when measurement values are present for different measuring signals, determining correlation value in said processor that represents a quality of correlation of the measurement values of the measuring signals with input parameters for the training data, with a measuring signal having a correlation value representing a best correlation then being used as the measurement signal for determining said displacement data.

15. A method as claimed in claim 14 comprising recording measuring signals continuously during recording of said training data for motion states that are not detected or that are combined from one time interval, and interpolating missing input parameters for comparison with said measurement values when determining the correlation values.

16. A method as claimed in claim 14 comprising forming a regression analysis to determine said assignment rule, and using a regression class of said regression algorithm as said correlation value.

17. A method as claimed in claim 1 comprising displacing said PET data in real time immediately after acquiring said PET data.

18. A method as claimed in claim 1 comprising determining a function of measured data events in said PET procedure in a predetermined data space as a sinogram for use as said measuring signal.

19. A method as claimed in claim 18 comprising using a plurality of measured PET events that occur in a spatially fixed volume of the patient in a single time interval as said measuring signal, and selecting said fixed volume from the group consisting of a slice of the patient and a PET image element, with respect to which accumulation of a PET tracer moves into and out of during said cyclical motion.

20. A method as claimed in claim 1 comprising operating said image recording facility to acquire said training data as a respiratory signal obtained from a source selected from the group consisting of a respiratory belt, a respiratory cushion, a three-dimensional camera, and a navigator scan executed by a magnetic resonance facility as said image recording facility.

21. An image recording system comprising:
a first image recording facility with a modality other than positron emission tomography (PET);
a control computer configured to operate said first image recording facility to acquire three-dimensional training data from a patient, exhibiting a cyclical motion, situated in the imaging recording facility, in a plurality of different motion states of the cyclical motion;
a processor provided with displacement data that describe a reference motion state, and said processor also being provided with said training data, and said processor being configured to determine model parameters of a statistical model that describes said cyclical motion, from deviations of said training data in said different motion states from said displacement data;
said processor being configured to determine an assignment rule for assigning measurement values of at least one measurement signal, which can be recorded during a PET measurement procedure and which exhibit said motion states of said cyclical motion, to input parameters that describe an instance of said statistical model;
a PET image recording facility;
said control computer being configured to operate said PET image recording facility to acquire PET data and to assign measurement values to the PET data acquired at respective recording points in time during a PET measurement procedure corresponding to the PET measurement procedure used to determine said rule;
said processor being configured to determine displacement data for said PET data by applying said assignment rule to said measurement values assigned to the PET data for said points in time; and
said processor being configured to spatially displace said PET data according to said displacement data to obtain corrected PET data in which location assignment errors due to said cyclical motion are compensated, and to make the corrected PET data available in electronic form, as a data file, from said processor.

22. A non-transitory, computer-readable data storage medium encoded with programming instructions, said storage medium being loaded into a control and processing computer system of an image recording system, and said programming instructions causing said control and processing computer system to:
operate an image recording facility with a modality other than PET to acquire three-dimensional training data from a patient, exhibiting a cyclical motion, situated in the imaging recording facility, in a plurality of different motion states of the cyclical motion;
receive displacement data that describe a reference motion state and receive said training data, and determine model parameters of a statistical model that describes said cyclical motion, from deviations of said training data in said different motion states from said displacement data;
determine an assignment rule for assigning measurement values of at least one measurement signal, which can be recorded during a PET measurement procedure and which exhibit said motion states of said cyclical motion, to input parameters that describe an instance of said statistical model;
operate a PET image recording facility to acquire PET data and to assign measurement values to the PET data acquired at respective recording points in time during a PET measurement procedure corresponding to the PET measurement procedure used to determine said rule;
determine displacement data for said PET data by applying said assignment rule to said measurement values assigned to the PET data for said points in time; and
spatially displace said PET data according to said displacement data to obtain corrected PET data in which location assignment errors due to said cyclical motion are compensated, and make the corrected PET data available in electronic form, as a data file, from said processor.
